(19) United States
(12) Patent Application Publication
(10) Pub. No.: US 2009/0118218 A1
(43) Pub. Date: May 7, 2009

(54) Biocompatible Hydrogels

(76) Inventor: Joshua Stopek, Yalesville, CT (US)

Correspondence Address: Tyco Healthcare Group LP, 60 MIDDLETOWN AVENUE, NORTH HAVEN, CT, 06473, US

(21) Appl. No.: 12/298,602

(22) PCT Filed: Jul. 11, 2007
(86) PCT No.: PCT/US07/15773

§ 371 Date: Oct. 27, 2008

Related U.S. Application Data
(60) Provisional application No. 60/819,964, filed on Jul. 11, 2006.

(51) Int. Cl. A61K 31/711; A61K 47/36; B32B 37/14; A61K 47/32

(52) U.S. Cl. 514/44; 514/777; 514/772.4; 156/326

(57) ABSTRACT

A biocompatible macromer composition is provided which includes a first polymer possessing a first nucleoside, and a second polymer possessing a second nucleoside that is complementary to the first nucleoside and capable of undergoing base pairing with the first nucleoside. The biocompatible macromer composition can be used as an adhesive or sealant in human and/or animal medical applications.

### CROSS-REFERENCE TO RELATED APPLICATIONS

This application claims the benefit of and priority to U.S. Provisional Patent Application No. 60/819,964 filed Jul. 11, 2006, the entire disclosure of which is incorporated by reference herein.

### TECHNICAL FIELD

The present disclosure relates to crosslinked compositions made from synthetic polymers and the use of such compositions as biological adhesives and/or sealants.

### BACKGROUND OF RELATED ART

In recent years there has developed increased interest in replacing or augmenting sutures with adhesive bonds. The reasons for this increased interest include: (1) the potential speed with which repair might be accomplished; (2) the ability of a bonding substance to effect complete closure, thus preventing seepage of fluids; and (3) the possibility of forming a bond without excessive deformation of tissue.

Studies in this area, however, have revealed that in order for surgical adhesives to be accepted by surgeons, they should possess a number of properties. They should exhibit high initial tack and an ability to bond rapidly to living tissue; the strength of the bond should be sufficiently high to cause tissue failure before bond failure; the adhesive should form a bridge, preferably a permeable flexible bridge; and the adhesive bridge and/or its metabolic products should not cause local histotoxic or carcinogenic effects.

Several materials useful as a tissue adhesive or tissue sealant are currently available. One type of adhesive that is currently available is a cyanoacrylate adhesive. However, there is the possibility that a cyanoacrylate adhesive can degrade to generate undesirable by-products such as formaldehyde. Another disadvantage is that cyanoacrylate adhesives can have a high flexural modulus which can limit the usefulness of the adhesive.

It would be desirable to provide a biological adhesive that is fully synthetic and therefore highly consistent in its properties without the concern of viral transmission. Such an adhesive should be flexible and biocompatible and should be suitable for use as either an adhesive or sealant.

### SUMMARY

The present disclosure provides biocompatible macromer compositions which may be utilized as adhesives or sealants to seal wounds or defects in tissue. In embodiments, the biocompatible macromer compositions include a first polymer including a first nucleoside and a second polymer including a second nucleoside, wherein the first nucleoside and the second nucleoside undergo base pairing thereby forming a biocompatible macromer composition of the present disclosure.

In embodiments, the molecular weight of the first polymer, and optionally the second polymer, may be from about 1×10³ g/mol to about 1×10⁶ g/mol. In other embodiments, the molecular weight of the first polymer, and optionally the second polymer, may be from about 1×10⁴ g/mol to about 1×10⁵ g/mol.

The present disclosure also provides adhesives for wound closure and sealants for use in medical applications which include the biocompatible macromer composition herein.

Methods for closing a wound with the biocompatible macromer composition of the present disclosure are also provided, wherein the biocompatible macromer composition is applied to the wound and allowed to set thereby closing said wound.

Methods for filling a void in animal tissue are also provided which include applying the biocompatible macromer composition of the present disclosure to the void and allowing the biocompatible macromer composition to set thereby filling the void.

Methods for adhering a medical device to a surface of animal tissue are also provided which include applying the biocompatible macromer composition of the present disclosure to the device, the surface of animal tissue, or both, bringing the device, biocompatible macromer composition and surface into contact with each other, and allowing the biocompatible macromer composition to set thereby adhering the device and surface to each other.

In other embodiments, the present disclosure provides methods for delivering a gene by administering the biocompatible macromer composition of the present disclosure to an animal, allowing the biocompatible macromer composition to set thereby forming a gel, and allowing the biocompatible macromer composition to degrade in vivo, wherein degradation of the biocompatible macromer composition delivers a gene to the animal.

### DETAILED DESCRIPTION

The biocompatible macromer compositions described herein may be useful as adhesives for adhering animal tissue or as sealants for sealing voids in animal tissue. The compositions of the present disclosure may be made using at least two components including polymeric chains terminated with nucleosides or nucleotides, including oligonucleotides or polynucleotides.

As used herein, “nucleoside” refers to a compound including a purine, deazapurine, or pyrimidine nucleobase, e.g., adenine, guanine, cytosine, uracil, thymine, deazaadenine, deazaguanosine, and the like, linked to a pentose at the 1′-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine.

“Nucleotide” refers to a phosphate ester of a nucleoside, e.g., a triphosphate ester, wherein the most common site of esterification is the hydroxyl group attached to the C-5 position of the pentose. A nucleotide may be composed of three moieties: a sugar, a phosphate, and a nucleobase. When part of a duplex, nucleotides are also referred to as “bases” or “base pairs”. The most common naturally-occurring nucleobases, adenine (A), guanine (G), uracil (U), cytosine (C), and thymine (T), bear the hydrogen-bonding functionality that effect base pairing.

The term “base pairing” refers to a pattern of specific pairs of nucleotides, and analogs thereof, that bind together through sequence-specific hydrogen-bonds, e.g. A pairs with T and U, and G pairs with C.

As used herein, an oligonucleotide may possess from about 1 to about 30 nucleotide units, while a polynucleotide may possess more than about 30 nucleotide units, in embodiments from about 30 to about 10,000 nucleotide units, in other embodiments from about 100 to about 7,500 nucleotide units, in yet other embodiments from about 1,000 to about 5,000 nucleotide units.

The polymers utilized to form the biocompatible macromer composition of the present disclosure possess nucleosides and/or nucleotides are selected to permit base pairing, for example: cytosine (C) bonding to guanine (G); and adenine (A) bonding to thymine (T) or uracil (U). The polymer chains may, in embodiments, possess additional functionalities including, but not limited to, acids, amines, sulfones, isocyanates, hydroxyl groups, vinyl groups, esters and the like. The additional functional groups may be selected to further enhance crosslinking between the first polymer chain and the second polymer chain.

It is contemplated that the macromer compositions described herein may be utilized both as adhesives and/or sealants that can be applied to living tissue and/or flesh of animals, including humans. While certain distinctions may be drawn between the usage of the terms “flesh” and “tissue” within the scientific community, the terms are used interchangeably herein as referring to a general substrate upon which those skilled in the art would understand the present adhesive to be utilized within the medical field for the treatment of patients. As used herein, “tissue” may include, but is not limited to, skin, bone, neuron, axon, cartilage, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic or ascite tissue.

In embodiments, a polymer chain utilized to form the macromer composition of the present disclosure may include the nucleoside or nucleotide itself, for example, the polymer chain may be oligodeoxyribonucleic acids such as oligodeoxyadenine, oligodeoxyguanine, oligodeoxycytosine, and oligodeoxythymidine; oligoribonucleic acids such as oligoadenine, oligoguanine, oligocytosine, oligouridine, and the like. The first polymer chain and the second polymer chain are selected to permit base pairing, for example, oligodeoxyguanine could be selected as the first polymer and oligodeoxycytosine could be selected as the second polymer; or oligodeoxyadenine could be selected as the first polymer and oligodeoxythymidine or oligouridine could be selected as the second polymer. In embodiments, where oligodeoxyadenine is selected as the first polymer, oligodeoxythymidine could be selected as the second polymer and oligouridine could be utilized as a third polymer, with base pairing occurring between the first polymer and the second polymer as well as between the first polymer and the third polymer.

In other embodiments, polynucleotides may be utilized as the first polymer chain and the second polymer chain, base pairing between the nucleobases occurring to form a biocompatible macromer composition of the present disclosure. Polynucleotides include, for example, polydeoxyribonucleic acids such as polydeoxyadenine, polydeoxyguanine, polydeoxycytosine and polydeoxythymidine.

In other embodiments, the polymer chain may include any suitable polymeric material for forming a hydrogel that may be functionalized with the complementary nucleosides and/or nucleotides. Such polymeric materials are within the purview of those skilled in the art and include, for example, hydrocarbons, polyalkylene oxides, polysaccharides, esters, aliphatic esters, orthoesters, phosphoesters, carbonates, urethanes, ethers, amides, phenolics, polymerized drugs, i.e., polyaspirin or polydiflunisal, commercially available from Polymerix Corp. (Piscataway, N.J.), combinations thereof, and the like, so long as the polymer selected is capable of being functionalized.

In embodiments, the polymeric materials may include absorbable materials including, but not limited to, glycolic acid, glycolide, lactic acid, lactide, 1,4-dioxane-2-one, 1,3-dioxane-2-one, ε-caprolactone, combinations thereof, and the like. In other embodiments, the polymer backbone may include a polysaccharide including, but not limited to, sorbitol, mannitol, sucrose, dextran, cyclodextrin, and the like. In yet other embodiments, the polymer backbone may include a functionalized PAO such as polyethylene glycol (“PEG”), polyethylene oxide (“PEO”), polypropylene oxide (“PPO”), a polyethylene glycol with lactide linkages, polypropylene glycol (“PPG”), co-polyethylene oxide lock or random copolymers, and poloxamers such as polyethylene oxide (PEO) copolymers with polypropylene oxide (PPO) such as the triblock PEO-PPO copolymers commercially available as PLURONICS® from BASF Corporation (Mt. Olive, N.J.).

In some embodiments, the first polymer may be a polyethylene oxide, such as a polyethylene glycol (“PEG”). As used herein, polyethylene glycol generally refers to a polymer with a molecular weight of less than 50,000, while polyethylene oxide is used to refer to a polymer having higher molecular weights. PEGs provide excellent water retention, flexibility and viscosity in the biocompatible adhesive composition.

In some embodiments the PEG may be modified to produce a multi-functional material, i.e., a branched or star configuration for improved biodegradability.

A first polymer backbone may be terminated with at least one nucleoside or nucleotide; in embodiments it may be terminated with two of the same nucleosides or nucleotides. Similarly a second polymer backbone may be terminated with at least one nucleoside or nucleotide; in embodiments it may be terminated with two of the same nucleosides or nucleotides, or even different nucleosides or nucleotides, so long as the nucleosides or nucleotides present on the second polymer backbone are complementary and capable of undergoing base pairing with the nucleosides or nucleotides located on the first polymer chain. Macromer compositions of the present disclosure may thus be formed by combining two or more of the polymer chains having complementary nucleosides or nucleotides, for example a first chain having cytosine with a second chain having guanine, or a first chain having adenine and a second chain possessing thymine or uracil.

The first polymer backbone may be present in the biocompatible macromer composition of the present disclosure in amounts from about 10% to about 90% by weight of the macromer composition, in embodiments from about 30% to about 70% by weight of the macromer composition, with the second polymer backbone present in amounts from about 90% to about 10% by weight of the macromer composition, in embodiments from about 70% to about 30% by weigh of the macromer composition.

As noted above, in embodiments, additional functional groups may be included on the backbone of the first and second polymer chains, either at one terminal, or along the chain itself. Suitable additional functional groups include, for example, acids, amines, sulfones, isocyanates, hydroxyl groups, vinyl groups, esters including N-hydroxy succinimide (NHS), combinations thereof, and the like. In embodiments, additional functional groups on the first polymer chain may be selected so that they are complementary to and may crosslink with additional functional groups on a second polymer chain. Thus, the polymer chain utilized to form the second polymer may possess a nucleoside or nucleotide complementary to the nucleoside or nucleotide located on the first polymer backbone, and the second polymer may also possess a second functional group reactive with the first functional group of the first polymer.

Examples of such additional complementary functional groups capable of crosslinking include, for example, amine or thiol groups reacting with succinimidyl groups; amino groups reacting with isocyanate groups; and the like. For example, where the first polymer backbone is functionalized to include additional amine groups, the second polymer backbone may be additionally functionalized to include amine-reactive groups, such as a succinimidyl group or an isocyanate group. Thus, where the first polymer possesses amine functional groups in addition to a nucleotide, the second polymer may also possess succinimidyl groups, isocyanate groups, or both, or diisocyanates such as ethylene diisocyanate, 1-6-hexamethylene diisocyanate (HMDI), 4,4′-oxybis(phenyl isocyanate), lysine diisocyanate, 2,4,6-trimethyl-1,3-phenylene diisocyanate, isophorone diisocyanate, cyclohexane-1,4-diisocyanate, 4,4′-dicyclohexylmethane diisocyanate, p-xylylene diisocyanate, tetramethylxylene diisocyanate, 1,4-phenylene diisocyanate, 2-4-toluene diisocyanate, 2,6-toluene diisocyanate, 4,4′-diphenylmethane diisocyanate, polymethylene polyphenyl polyisocyanates, 2,4′-diphenylmethane diisocyanate, 3(4)-isocyanatomethyl-1-methyl cyclohexyl isocyanate, 1,5-naphthylene diisocyanate, and mixtures and combinations thereof.

The presence of these additional functional groups in addition to the complementary nucleosides or nucleotides may further enhance crosslinking of the first and second polymers to form a macromer composition of the present disclosure, thereby forming a hydrogel suitable for use as an adhesive or sealant.

The preparation of substituted polymers is within the purview of those skilled in the art. For example, polymers such as PEG can be functionalized, according to any method within the purview of those skilled in the art including, for example, those methods disclosed in Chapter 22 of Poly(ethylene Glycol) Chemistry: Biotechnical and Biomedical Applications, J. Milton Harris, ed., Plenum Press, NY (1992). Various forms of PAOs, including functionalized PEGs, are also commercially available from providers which include, for example, Shearwater Polymers, Inc., Huntsville, Ala., and Texaco Chemical Company, Houston, Tex. PEGs are commercially available from a variety of sources, including Nektar Therapeutics, 150 Industrial Road, San Carlos, Calif., USA 94070.

The polymer backbones utilized to form the biocompatible macromer composition of the present disclosure should have a molecular weight sufficiently high so that, when the first polymer backbone is crosslinked with the second polymer backbone, the resulting macromer composition provides adequate adhesive or sealant properties. At the same time, the molecular weight of the polymer backbones should be sufficiently low so that, upon degradation, the resulting polymer fragments can be excreted by the body. The molecular weight of the first and second polymer backbones can be from about 1×10³ g/mol to about 1×10⁶ g/mol, in embodiments from about 1×10⁴ g/mol to about 1×10⁵ g/mol.

In some embodiments, the backbone of the first polymer and/or second polymer can have a branched or multi-arm structure. For example, a polyalkylene oxide backbone can be the result of polymerizing an alkylene oxide monomer in the presence of a multi-functional (e.g., polyhydric) initiator. Reaction conditions for producing branched or multi-arm polyalkylene oxide backbones are within the purview of those skilled in the art.

The degree of substitution of the polymer will be a factor in the amount of crosslinking ultimately achieved and thus in the flexibility and swelling of the macromer composition of the present disclosure. The crosslinking between the first polymer and the second polymer can occur via hydrogen bonds and/or hydrophobic bonds.

Where the polymer backbones are based upon polyalkylene oxides, the nucleosides or nucleotides and any additional functional groups may be terminally located on the polyalkylene oxide chains or, alternatively, located at one or more location along the polyalkylene oxide chains. Where the polyalkylene oxide is of a branched or dendrimeric configuration, the nucleosides or nucleotides and any additional functional groups may be terminally located on the polyalkylene oxide arms or, alternatively, located at one or more location along the polyalkylene oxide arms. Similarly, although a single nucleoside or nucleotide group may be present per polyalkylene oxide chain or arm, it is also contemplated that more than one and up to ten or more nucleosides or nucleotides and/or any additional functional groups per polyalkylene oxide chain or arm may be present.

The selection of the components of the macromer compositions of the present disclosure can be adjusted to tailor the macromer composition for optimal viscosity according the desired adhesive and/or sealant use. The viscosity of the macromer composition of the present disclosure should be sufficient to weld tissue yet still degrade in the body. Higher viscosities minimize displacement of the adhesive and/or sealant. Higher viscosities also improve the retention of uncured or unpolymerized adhesives and/or sealants at the site of application. These higher viscosities will, however, make the macromer compositions more difficult to apply. A useful viscosity for an adhesive and/or sealant utilizing a macromer composition of the present disclosure may be from about 200 centipoise (“cP”) to about 100,000 cP, in embodiments from about 500 to about 10,000 cP.

Optionally, at least one additional component providing hydrolytically degradable bonds can be incorporated into the first polymer, the second polymer, or both, thereby increasing the rate at which the macromer composition of the present disclosure degrades. Suitable components which can be optionally incorporated include, but are not limited to, hydrolytically labile α-hydroxy acids (such as, for example, lactic acid or glycolic acid), esters (such as, for example, lactide or glycolide), lactones (such as, for example, ε-caprolactone), carbonates (such as, for example, trimethylene carbonate), ester ethers (such as, for example, dioxanones such as 1,4-dioxane-2-one or 1,3-dioxane-2-one), diacids (such as, for example, succinnic acid, adipic acid, sebacic acid, malonic acid, glutaric acid, etc.), and combinations thereof. Those skilled in the art will readily envision reaction schemes for incorporating these components into the first polymer, the second polymer, or both.

For example, where the first polymer and second polymer are based on polyalkylene oxides, these hydrolytically degradable components can be incorporated into the first and/or second polyalkylene oxide by reacting both components with small amounts of diol. In embodiments a low weight PEG polymer may be combined with a diol mixture. The diol mixture results in degradable ester links between the highly branched polymer chains. A very low diol concentration should be used to prevent the polymer from gelling prematurely. The selected diol may be chosen according to the desired properties of the final sealant. For example, where mechanical enhancement is not desired or necessary, propylene fumarate, diethylene glycol or a short chain PEG diol can be used. Where additional strength of the sealant is desired, phthalic, biphenyl, bisphenol A, or a diglycidyl ether of bisphenol A groups can be used.

In addition to or in place of components that provide hydrolytically degradable linkages, at least one linkage that is enzymatically degradable may be incorporated into the first polymer, the second polymer, or both. Linkages which are enzymatically degradable include, but are not limited to: an amino acid residue such as -Arg-, -Ala-, -Ala(D)-, -Val-, -Leu-, -Lys-, -Pro-, -Phe-, -Tyr-, -Glu-, and the like; 2-mer to 6-mer oligopeptides such as -Ile-Glu-Gly-Arg-, -Ala-Gly-Pro-Arg-, -Arg-Val-(Arg)₂-, -Val-Pro-Arg-, -Gln-Ala-Arg-, -Gln-Gly-Arg-, -Asp-Pro-Arg-, -Gln(Arg)₂-, Phe-Arg-, -(Ala)₃-, -(Ala)₂-, -Ala-Ala(D)-, -(Ala)₂-Pro-Val-, -(Val)₂-, -(Ala)₂-Leu-, -Gly-Leu-, -Phe-Leu-, -Val-Leu-Lys-, -Gly-Pro-Leu-Gly-Pro-, -(Ala)₂-Phe-, -(Ala)₂-Tyr-, -(Ala)₂-His-, -(Ala)₂-Pro-Phe-, -Ala-Gly-Phe-, -Asp-Glu-, -(Glu)₂ -, -Ala-Glu-, -Ile-Glu-, -Gly-Phe-Leu-Gly-, -(Arg)₂-; D-glucose, N-acetylgalactosamine, N-acetylneuraminic acid, N-acetylglucosamine, N-acetylmannosamine or the oligosaccharides thereof; oligodeoxyribonucleic acids such as oligodeoxyadenine, oligodeoxyguanine, oligodeoxycytosine, and oligodeoxythymidine; oligoribonucleic acids such as oligoadenine, oligoguanine, oligocytosine, oligouridine, and the like. Those skilled in the art will readily envision reaction schemes for incorporating enzymatically degradable linkages into the polymer.

A variety of optional ingredients may be included in the macromer composition of the present disclosure. A phospholipid surfactant that provides antibacterial stabilizing properties and helps disperse other materials in the macromer composition of the present disclosure may be added. Optional additives include antimicrobial agents, colorants, preservatives, or medicinal agents such as, for example, protein and peptide preparations, antipyretic, antiphlogistic and analgesic agents, anti-inflammatory agents, vasodilators, antihypertensive and antiarrhythmic agents, hypotensive agents, antitussive agents, antineoplastics, local anesthetics, hormone preparations, antiasthmatic and antiallergic agents, antihistaminics, anticoagulants, antispasmodics, cerebral circulation and metabolism improvers, antidepressant and antianxiety agents, vitamin D preparations, hypoglycemic agents, antiulcer agents, hypnotics, antibiotics, antifungal agents, sedative agents, bronchodilator agents, antiviral agents, dysuric agents, and polymeric drugs such as polyaspirin, polydiflunisal, polytriclosan, polychlorhexidine, and the like. Such optional ingredients may, in embodiments, be pendant, back-bone derivatives.

Additionally, an enzyme may be added to the macromer composition of the present disclosure to increase the rate of degradation of the macromer composition. Suitable enzymes include, for example, peptide hydrolases such as elastase, cathepsin G, cathepsin E, cathepsin B, cathepsin H, cathepsin L, trypsin, pepsin, chymotrypsin, γ-glutamyltransferase (γ-GTP) and the like; sugar chain hydrolases such as phosphorylase, neuraminidase, dextranase, amylase, lysozyme, oligosaccharase and the like; oligonucleotide hydrolases such as alkaline phosphatase, endoribonuclease, endodeoxyribonuclease, and the like. Enzymes may be added in liposomes or microspheres to control their release and thus their effect on the degradation of the macromer composition of the present disclosure. Methods for incorporating enzymes into liposomes and microspheres are within the purview of those skilled in the art.

The macromer compositions of the present disclosure can be used in human and animal medical applications including, but not limited to, wound closure (including surgical incisions and other wounds), adhesives for medical devices (including implants), sealants and void fillers, gene delivery devices, drug delivery devices, and embolic agents. As used herein, an adhesive or sealant including a macromer composition of the present disclosure may be used for any of the above-described medical applications as well as any other medical application within the purview of those skilled in the art.

In some embodiments, the first and second polymer components may be kept separate prior to application to tissue. Thus, the first and second polymer components can be dispensed from a conventional two-part adhesive dispenser which provides mixing of the two components either prior to or after leaving the dispenser. Such dispensers are disclosed, for example, in U.S. Pat. Nos. 4,978,336, 4,361,055, 4,979,942, 4,359,049, 4,874,368, and 5,368,563, the disclosures of which are incorporated herein by reference.

In other embodiments, especially where the macromer composition of the present disclosure is to be utilized as a void filler or sealant to fill a defect in an animal's body, it may be advantageous to more precisely control the conditions and extent of cross-linking; in such a case, it may be useful to partially cross-link the macromer composition prior to its use to fill a void in animal tissue. In such a case the macromer composition of the present disclosure is applied to the void or defect and allowed to set, thereby filling the void or defect.

The macromer composition of the present disclosure can be used for a number of different applications. These applications include use as an adhesive to bind tissue together either as a replacement of, or as a supplement to, sutures, staples, tapes and/or bandages. Use of the disclosed macromer composition as an adhesive can eliminate or substantially reduce the number of sutures normally required during current practices, and eliminate the subsequent need for removal of staples and certain types of sutures. The use of macromer compositions of the present disclosure as an adhesive thus can be particularly useful with delicate tissues where sutures, clamps or other conventional tissue closure mechanisms may cause further tissue damage.

Additional applications for macromer compositions of the present disclosure include sealing tissues to prevent or control blood, or other fluid leaks, at suture or staple lines. In another embodiment, the macromer composition can be used to attach skin grafts and position tissue flaps during reconstructive surgery. In still another embodiment, the macromer composition can be used to close tissue flaps in periodontal surgery.

To effectuate the joining of two tissue edges, the two edges are approximated, and the first polymer with a first nucleotide is combined with the second polymer having a complementary nucleotide. The crosslinking reaction is rapid, generally taking less than one minute. Thus, the macromer composition of the present disclosure can be used as an adhesive to close a wound, including a surgical incision. In such a case, the macromer composition of the present disclosure can be applied to the wound and allowed to set, thereby closing the wound.

In embodiments, it may be useful to add a coupling agent to the first polymer, the second polymer, or both during formation of the macromer composition of the present disclosure to further enhance crosslinking of functional groups present in addition to the nucleosides or nucleotides. Suitable coupling agents are within the purview of those skilled in the art. In embodiments, a carbodiimide may be utilized as a coupling agent. Specific carbodiimides which may be utilized include, but are not limited to, 1-ethyl-3(3-dimethyl-aminopropyl)-carbodiimide hydrochloride.

In another embodiment, the present disclosure is directed to a method for using the macromer composition of the present disclosure to adhere a medical device to tissue, rather than secure two edges of tissue. In some cases a coating may be present on the medical device which can include the first polymer component, the second polymer component, or both. In some aspects, the medical device includes an implant. Other medical devices include, but are not limited to, pacemakers, stents, shunts and the like. Generally, for adhering a device to the surface of animal tissue, the macromer composition of the present disclosure, or components thereof, can be applied to the device, the tissue surface or both. The device, macromer composition and tissue surface are then brought into contact with each other and the composition is allowed to set, thereby adhering the device and surface to each other.

The macromer composition of the present disclosure can also be used to prevent post surgical adhesions. In such an application, the macromer composition may be applied and cured as a layer on surfaces of internal tissues in order to prevent the formation of adhesions at a surgical site during the healing process.

When used as a sealant, the macromer composition of the present disclosure can be used in surgery to prevent or inhibit bleeding or fluid leakage both during and after a surgical procedure. It can also be applied to prevent air leaks associated with pulmonary surgery. The sealant may be applied directly to the desired area in at least an amount necessary to seal off any defect in the tissue and seal off any fluid or air movement.

Application of the macromer composition as an adhesive or sealant, with or without other additives, can be done by any conventional means. These include dripping, brushing, or other direct manipulation of the adhesive on the tissue surface, or spraying of the adhesive to the surface. In open surgery, application by hand, forceps or the like is contemplated. In endoscopic surgery, the adhesive can be delivered through the cannula of a trocar, and spread at the site by any device known in the art.

The biocompatible macromer composition of the present disclosure may also, in embodiments, be used as a gene delivery system. As the first polymer and second polymer may be made of oligonucleotides or polynucleotides, in embodiments the first polymer and second polymer may be selected not only for their ability to undergo base pairing, but they may also be selected so that they encode a gene of interest for administration to an animal as part of gene therapy. Once formed, the biocompatible macromer composition of the present disclosure degrades in vivo, thereby releasing the gene in the subject animal. The addition or enzymes of the inclusion of degradable linkages in the first polymer, the second polymer, or both, may further enhance the degradation and thus the ability of a biocompatible macromer composition to deliver a desired gene in vivo and thus function as a gene delivery system.

It will be understood that various modifications may be made to the embodiments disclosed herein. Therefore the above description should not be construed as limiting, but merely as exemplifications of some embodiments. Those skilled in the art will envision other modifications within the scope and spirit of the claims appended hereto.

What is claimed is:

1. A biocompatible macromer composition comprising:
a first polymer comprising a first nucleoside; and
a second polymer comprising a second nucleoside,
wherein the first nucleoside and the second nucleoside undergo base pairing, and the composition acts as an adhesive, sealant, or gene delivery device.

2. A biocompatible macromer composition as in claim 1, wherein the first polymer comprises an oligonucleotide.

3. A biocompatible macromer composition as in claim 1, wherein the second polymer comprises an oligonucleotide.

4. A biocompatible macromer composition as in claim 1, wherein the first polymer comprises a polynucleotide.

5. A biocompatible macromer composition as in claim 1, wherein the second polymer comprises a polynucleotide.

6. A biocompatible macromer composition as in claim 1, wherein the molecular weight of the first polymer and optionally the second polymer is from about 1×10³ g/mol to about 1×10⁶ g/mol.

7. A biocompatible macromer composition as in claim 1, wherein the molecular weight of the first polymer and optionally the second polymer is from about 1×10⁴ g/mol to about 1×10⁵ g/mol.

8. A biocompatible macromer composition as in claim 1, wherein the first polymer further comprises a polymer selected from the group consisting of polyalkylene oxides, polysaccharides, esters, aliphatic esters, orthoesters, phosphoesters, carbonates, urethanes, ethers, amides, phenolics, and combinations thereof.

9. A biocompatible macromer composition as in claim 1, wherein the second polymer further comprises a polymer selected from the group consisting of polyalkylene oxides, polysaccharides, esters, aliphatic esters, orthoesters, phosphoesters, carbonates, urethanes, ethers, amides, phenolics, and combinations thereof.

10. A biocompatible macromer composition as in claim 1, wherein the first polymer further comprises a first functional group selected from the group consisting of acids, amines, sulfones, isocyanates, hydroxyl groups, vinyl groups, esters, and combinations thereof, and the second polymer further comprises a second functional group that crosslinks with the first functional group.

11. A biocompatible macromer composition as in claim 1, wherein the first polymer and optionally the second polymer further comprises bioabsorbable groups selected from the group consisting of α-hydroxy acids, esters, lactones, carbonates, ester ethers, diacids, and combinations thereof.

12. A biocompatible macromer composition as in claim 1, wherein the first polymer and optionally the second polymer further comprises bioabsorbable groups selected from the group consisting of lactic acid, glycolic acid, lactide, glycolide, ε-caprolactone, trimethylene carbonate, 1,4-dioxane-2-one, 1,3-dioxane-2-one, succinnic acid, adipic acid, sebacic acid, malonic acid, glutaric acid, and combinations thereof.

13. A biocompatible macromer composition as in claim 1, wherein the first polymer is present in an amount from about 10% to about 90% by weight of the synthetic macromer composition.

14. A biocompatible macromer composition as in claim 1, wherein the second polymer is present in an amount from about 90% to about 10% by weight of the synthetic macromer composition.

15. A biocompatible macromer composition as in claim 1, wherein the first polymer and optionally the second polymer is combined with enzymatically degradable components.

16. An adhesive for wound closure comprising the biocompatible macromer composition of claim 1.

17. A sealant for use in a medical application comprising the biocompatible macromer composition of claim 1.

18. A method for closing a wound comprising:
applying the biocompatible macromer composition of claim 1 to said wound; and
allowing the biocompatible macromer composition to set thereby closing said wound.

19. The method of claim 18, wherein the wound is a surgical incision.

20. A method for filling a void in animal tissue comprising:
applying the biocompatible macromer composition of claim 1 to said void; and
allowing the biocompatible macromer composition to set thereby filling said void.

21. A method for adhering a medical device to a surface of animal tissue comprising the steps of:
applying the biocompatible macromer composition of claim 1 to said device, said surface or both;
bringing the device, biocompatible macromer composition and surface into contact with each other; and
allowing the biocompatible macromer composition to set thereby adhering the device and surface to each other.

22. The method of claim 21 wherein said medical device is an implant.

23. A method for delivering a gene comprising:
administering the biocompatible macromer composition of claim 1 to an animal;
allowing the biocompatible macromer composition to set thereby forming a gel; and
allowing the biocompatible macromer composition to degrade in vivo, wherein degradation of the biocompatible macromer composition delivers a gene to the animal.
